(19) United States
(12) Patent Application Publication
(10) Pub. No.: US 2026/0250306 A1
(43) Pub. Date: Aug. 27, 2026

(54) COMPOUND AND ANTISTATIC AGENT CONTAINING THE SAME AS ACTIVE COMPONENT

(71) Applicant: NIPPON CHEMICAL INDUSTRIAL CO., LTD., Tokyo (JP)

(72) Inventors: Masashi Sugiya, Tokyo (JP); Eri Furui, Tokyo (JP)

(73) Assignee: NIPPON CHEMICAL INDUSTRIAL CO., LTD., Tokyo (JP)

(21) Appl. No.: 18/872,449

(22) PCT Filed: Jun. 7, 2023
(86) PCT No.: PCT/JP2023/021158

§ 371 Date: Dec. 6, 2024

(30) Foreign Application Priority Data
Jun. 9, 2022 (JP) 2022-093585
Jun. 2, 2023 (JP) 2023-091845

(51) Int. Cl. C07F 9/54

(52) U.S. Cl. CPC C07F 9/5407

(57) ABSTRACT

To provide a suitable compound as an antistatic agent that has an excellent compatibility with a resin and is stably fixed on a surface of the resin, and can develop a high antistatic performance even in a small amount. The present invention is to provide a compound represented by General Formula (1) below: (where R¹, R², and R³ each independently represent a straight-chain or branched-chain alkyl group having 1 to 20 carbon atoms, R⁴ represents a straight-chain or branched-chain fluoroalkyl group having 1 to 20 carbon atoms, and A represents an anion having a fluorine atom.)

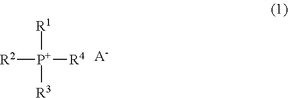

### TECHNICAL FIELD

The present invention relates to a compound that is a phosphonium salt having a fluoroalkyl group and an antistatic agent containing the same as an active component.

### BACKGROUND ART

So far, various methods for preventing electrostatic damage have been proposed, but in many cases, the problems have been solved by using antistatic agents. As the antistatic agents, surfactants, conductive fillers, conductive polymers, ion liquids, and the like are used. Among them, the development of the antistatic performance of ion liquids is different from the mechanisms of other antistatic agents. For example, when an ion liquid is used as an antistatic agent of a resin, the static electricity generated on the surface of the resin is not easily affected by external environments such as temperature and humidity because the antistatic performance is developed by neutralizing the charges of static electricity due to the bias in the concentration of the ion liquid in the resin. In addition, since molecular design can be achieved by combining positive and negative ions, there are advantages such as ease of adjustment of compatibility with the resin and excellent appearance properties such as keeping transparency.

There are various proposals for technologies using ion liquids as antistatic agents, depending on the purpose. For example, Patent Literature 1 discloses an antistatic agent containing an inclusion compound in which an ion liquid is included in cyclodextrin as an antistatic agent with excellent water resistance and solvent resistance, and describes a specific ammonium salt or phosphonium salt as an ion liquid that can be included in cyclodextrin. Patent Literature 2 discloses an antistatic agent that is easily miscible with hydrocarbons by containing an anion of docusate, the so-called bis(2-ethylhexyl) ester of sulfone succinic acid, and describes that it can be used as an antistatic additive for fuels and polymers. Furthermore, Patent Literature 3 describes that a compound, which contains a polymerizable anion having a specific structure including a perfluoroalkyl group and a polymerizable functional group in the molecule, and a monovalent cation, is used in the form of a polymerizable ion liquid, as an antistatic agent with excellent voltage resistance characteristics.

### CITATION LIST

### Patent Literature

Patent Literature 1: Japanese Patent Laid-Open No. 2017-52859
Patent Literature 2: Japanese Translation of PCT International Application Publication No. 2006-510581
Patent Literature 3: International Publication No. WO 2020/031836

### SUMMARY OF INVENTION

### Technical Problem

As described above, the antistatic performance imparted by ion liquids is not easily affected by the external environment. Therefore, if the compatibility with resins can be improved, the ion liquids can be expected to be used as permanent antistatic agents. However, because resins have very high insulating properties, they are extremely charged, and this charging can easily cause the resin surface to become contaminated due to the adsorption of dust and the like. Therefore, in order to continuously obtain antistatic properties, it is necessary to stably fix the ion liquid on the resin surface.

However, the antistatic agents using ion liquids that have been conventionally proposed have excellent antistatic properties, but stable fixing on the resin surface is difficult. As one method for solving this problem, use of a polymerizable ion liquid as described in Patent Literature 3 makes it possible to increase the contact area with the resin to thereby achieve fixing. However, a large amount of the ion liquid is required in order to increase the antistatic performance, which causes problems such as impairing the properties and the appearance required for the resins.

Therefore, an object of the present invention is to provide a suitable compound as an antistatic agent that has an excellent compatibility with a resin and is stably fixed on a surface of the resin, and can develop a high antistatic performance even in a small amount.

### Solution to Problem

As a result of extensive research in light of the above-described circumstances, the present inventors found that when a functional group is incorporated into a phosphonium cation and an anion having a fluorine atom is combined therewith in a phosphonium-type ion liquid, the ion liquid is stably fixed on the resin surface, and therefore excellent permanent antistatic properties are exhibited even in small amounts, and the present inventors completed the present invention.

That is, the present invention is to provide a compound represented by General Formula (1) below:

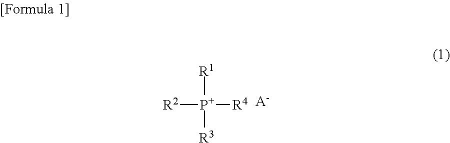

where R¹, R², and R³ each independently represent a straight-chain or branched-chain alkyl group having 1 to 20 carbon atoms, R⁴ represents a straight-chain or branched-chain fluoroalkyl group having 1 to 20 carbon atoms, and A represents an anion having a fluorine atom.

### Advantageous Effects of Invention

According to the present invention, it is possible to provide a suitable compound as an antistatic agent that has an excellent compatibility with a resin and is stably fixed on a surface of the resin, and can develop a high antistatic performance even in a small amount.

### DESCRIPTION OF EMBODIMENTS

A compound of the present invention is a phosphonium salt having a fluoroalkyl group, represented by the following General Formula (1).

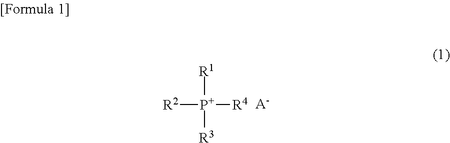

In the General Formula (1), R¹, R², and R³ represent a straight-chain or branched-chain alkyl group having 1 to 20 carbon atoms, preferably 2 to 18 carbon atoms, and particularly preferably 3 to 15 carbon atoms. R¹, R², and R³ each may be the same or different, but is preferably the same from the viewpoint of synthesis.

R⁴ in General Formula (1) represents a straight-chain or branched-chain fluoroalkyl group having 1 to 20 carbon atoms, preferably 2 to 18 carbon atoms, and particularly preferably 3 to 15 carbon atoms. In addition, A represents an anion having a fluorine atom.

Specific examples of the straight-chain alkyl group having 1 to 20 carbon atoms represented by R¹, R², and R³ include a methyl group, an ethyl group, a n-propyl group, a n-butyl group, a n-pentyl group, a n-hexyl group, a n-heptyl group, a n-octyl group, a n-nonyl group, a n-decyl group, a n-undecyl group, a n-tridecyl group, a n-tetradecyl group, a n-pentadecyl group, a n-hexadecyl group, a n-heptadecyl group, a n-octadecyl group, a n-nonadecyl group, and a n-icosyl group.

Specific examples of the branched-chain alkyl group having 1 to 20 carbon atoms represented by R¹, R², and R³ include an isopropyl group, an isobutyl group, a s-butyl group, a t-butyl group, an isopentyl group, a s-pentyl group, a t-pentyl group, an isohexyl group, a s-hexyl group, a t-hexyl group, and an ethylhexyl group.

Examples of the fluoroalkyl group having 1 to 20 carbon atoms represented by R⁴ include a group obtained by substituting, with a fluorine atom, at least one hydrogen atom in the straight-chain or branched-chain alkyl group having 1 to 20 carbon atoms.

As the fluoroalkyl group having 1 to 20 carbon atoms represented by R⁴, a group represented by the following General Formula (2) is particularly preferable because compatibility and fixation with a resin can be achieved.

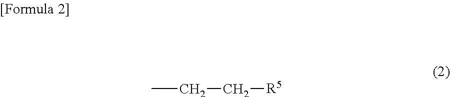

where R⁵ represents a straight-chain or branched-chain perfluoroalkyl group having 1 to 18 carbon atoms.

Examples of the straight-chain or branched-chain perfluoroalkyl group having 1 to 18 carbon atoms include a trifluoromethyl group, a perfluoroethyl group, a perfluoropropyl group, a perfluorobutyl group, a perfluoropentyl group, a perfluorohexyl group, a perfluoroheptyl group, a perfluorooctyl group, and a perfluorodecyl group.

A in the General Formula (1) represents an anion having a fluorine atom. Examples thereof include tetrafluoroborate (BF₄), hexafluorophosphate (PF₆), bis(trifluoromethanesulfonyl)imide (N(SO₂CF₃)₂), bis(fluorosulfonyl)imide (N(SO₂F)₂), trifluoromethanesulfonate (SO₃CF₃), tris(pentafluoroethyl) trifluorophosphate ((C₂F₅)₃ PF₃), and trifluoroacetic acid (CF₃COO). Among them, bis(fluorosulfonyl)imide (N(SO₂F)₂) and bis(trifluoromethanesulfonyl)imide (N(SO₂CF₃)₂) are preferable because compatibility and fixation with a resin can be achieved.

Specific examples of the compound of the present invention represented by General Formula (1) include tributyl(1H, 1H, 2H, 2H-heptadecafluorodecyl)phosphonium·bis(trifluoromethanesulf onyl)imide, tributyl(1H, 1H, 2H, 2H-heptadecafluorodecyl)phosphonium·bis(fluorosulfonyl)imide, tributyl(1H, 1H, 2H, 2H-nonafluorohexyl)phosphonium·bis(trifluoromethanesulfonyl)imide, tributyl(1H, 1H, 2H, 2H-nonafluorohexyl)phosphonium·bis(fluorosulfonyl)imide, tributyl(1H, 1H, 2H, 2H-tridecafluoro-n-octyl)phosphonium·bis(trifluoromethanesulfonyl)imide, triethyl(1H, 1H, 2H, 2H-heptadecafluorodecyl)phosphonium·bis(trifluoromethanesulf onyl)imide, triethyl(1H, 1H, 2H, 2H-heptadecafluorodecyl)phosphonium·bis(fluorosulfonyl)imide, triethyl(1H, 1H, 2H, 2H-nonafluorohexyl)phosphonium·bis(trifluoromethanesulfonyl)imide, triethyl(1H, 1H, 2H, 2H-nonafluorohexyl)phosphonium·bis(fluorosulfonyl)imide, triethyl(1H, 1H, 2H, 2H-tridecafluoro-n-octyl)phosphonium·bis(trifluoromethanesulfonyl)imide, trioctyl(1H, 1H, 2H, 2H-heptadecafluorodecyl)phosphonium·bis(trifluoromethanesulf onyl)imide, trioctyl(1H, 1H, 2H, 2H-heptadecafluorodecyl)phosphonium·bis(fluorosulfonyl)imide, trioctyl(1H, 1H, 2H, 2H-nonafluorohexyl)phosphonium·bis(trifluoromethanesulfonyl)imide, trioctyl(1H, 1H, 2H, 2H-nonafluorohexyl)phosphonium·bis(fluorosulfonyl)imide, and trioctyl(1H, 1H, 2H, 2H-tridecafluoro-n-octyl)phosphonium·bis(trifluoromethanesulfonyl)imide.

The compound represented by the General Formula (1) is obtained in the following manner. For example, trialkylphosphine is allowed to react with halogenated fluoroalkyl in an inert gas atmosphere, to obtain a phosphonium salt in which an anion is halogen. Then, neutralization/oxidation treatment is performed if desired, and a desired anion component is added dropwise to the obtained phosphonium salt, followed by mixing. Then, the resultant is subjected to anion exchange, to obtain the compound represented by the General Formula (1). The compound represented by the General Formula (1) obtained after the anion exchange can be used as an active component of an antistatic agent that will be described later by undergoing steps such as washing, concentration, drying, and pulverization if desired.

The antistatic agent of the present invention is characterized by an active component of a phosphonium salt that is the compound represented by the General Formula (1). As a similar cationic antistatic agent, an ammonium salt or a sulfonium salt is also known, but it is known that the phosphonium salt has a higher heat resistance than other onium salts, can be kneaded and molded with a resin at a relatively high temperature, and is not thermally decomposed. Therefore, even in cases where other onium salts cannot be used due to thermal decomposition, phosphonium salts can be used without any problems.

When three of four substituents of the cation moiety in the compound represented by the General Formula (1) are relatively short alkyl groups having 1 to 8 carbon atoms and the other is a long-chain fluoroalkyl group having 1 to 20 carbon atoms, the compound has an excellent antistatic performance and is stably fixed on a resin. In addition, when the anion moiety has a fluorine atom, compatibility and fixation with a resin can be achieved.

The antistatic agent of the present invention can be either water-soluble or oil-soluble depending on the physical properties of the compound represented by the General Formula (1). When the antistatic agent is used, resin additives such as an antioxidant, an ultraviolet inhibitor, a weather-resistant agent, an anti-blocking agent, a pigment, a reinforcing agent, a lubricant, a plasticizer, or other antistatic agents may be used in combination if necessary.

The antistatic agent of the present invention is solid or liquid at room temperature. When the antistatic agent of the present invention is solid at room temperature, it is preferably in the form of powder because the antistatic agent can be easily liquefied by heating when kneaded with a resin and is able to be uniformly dispersed. The average particle size determined by the laser diffraction scattering method is preferably 100 μm or less, more preferably 0.1 to 50 μm or less, and particularly preferably 0.5 to 20 μm.

Furthermore, when the antistatic agent of the present invention is liquid at room temperature, it is preferable that the viscosity at 25° C. is 10 to 300 cP, and particularly 15 to 250 cP because the antistatic agent can be uniformly dispersed when kneaded with a resin, to thereby obtain an excellent antistatic effect. The viscosity at 25° C. can be measured by a rotatory viscometer, a vibration viscometer, or the like.

The material into which the antistatic agent of the present invention is incorporated is not particularly limited as long as the material is electrostatically charged. Examples thereof include polymer materials such as synthetic resins or rubber and molded bodies thereof, fibers, films, nonwoven fabrics, and beads.

The type of polymer material to which the antistatic agent of the present invention can be applied is not particularly limited. Examples thereof include: polyolefins such as polyethylene, polypropylene, and polystyrene and copolymers thereof; thermoplastic resins such as polycarbonate, polyethylene terephthalate, polymethyl methacrylate, polybutylene terephthalate, polycaprolactam, amide-based resins, phenylene oxide-based resins, vinyl-based resins, acetal-based resins, ketone-based resins, sulfide-based resins, and urethane-based resins, and copolymers thereof; and fluorine-based resins obtained by fluorinating these polymer materials. Particularly, application to the fluorine-based resin makes it possible to obtain advantages such as compatibility and fixation with a resin.

Examples of the fluorine-based resin include polytetrafluoroethylene, polyhexafluoropropylene, polyvinylidene fluoride, polyvinyl fluoride, perfluoroalkoxyalkane, polychlorotrifluoroethene, (tetrafluoroethylene/hexafluoropropylene) copolymer, (tetrafluoroethylene/perfluoroalkyl vinyl ether) copolymer, (ethylene/tetrafluoroethylene) copolymer, (propylene/hexafluoropropylene) copolymer, (vinylidene fluoride/ethylene) copolymer, and (vinylidene fluoride/tetrafluoroethylene/hexafluoropropylene) copolymer.

Examples of the rubber include styrene·butadiene rubber, butadiene rubber, isoprene rubber, chloroprene rubber, butyl rubber, urethane rubber, silicone rubber, NBR, EPM, hydrogenated nitrile rubber, polysulfide rubber, acrylic rubber, ethylene acrylic rubber, and fluorine-based rubbers obtained by fluorinating these rubbers.

Examples of the fluorine-based rubbers include tetrafluoroethylene-based rubber, tetrafluoroethylenepropylene-based rubber, vinylidene fluoride-based rubber, hexafluorobutadiene-based rubber, fluorosilicone-based rubber, tetrafluoroethylene-propylene-based rubber, and tetrafluoroethylene-perfluoroalkyl vinyl ether-based rubber.

A method for applying the antistatic agent of the present invention to the polymer material may be any method. Examples thereof include a method for adding the antistatic agent internally to a desired polymer material and a method for coating the antistatic agent to the surface of a molded body formed of a polymer material.

Examples of the method for adding the antistatic agent of the present invention internally to the polymer include a method for adding and mixing the antistatic agent of the present invention during processing or production of the polymer. Examples of the method for adding the antistatic agent of the present invention during processing of the polymer include: directly adding the antistatic agent of the present invention to a desired polymer material, mixing the resultant with a tumbler, a ribbon blender, a high-speed mixer, or the like, and melt-mixing the resultant so that the antistatic agent is uniformly distributed in the polymeric material; or adding the antistatic agent of the present invention in the form of master chips in which the antistatic agent is contained at a high concentration, followed by melt-mixing. The antistatic agent of the present invention can also be added and mixed before or during the polymerization process of polyethylene terephthalate, or the like.

The blending amount of the antistatic agent of the present invention when the antistatic agent is internally added to the polymer material is normally 0.01 to 10% by mass, and preferably 0.1 to 5% by mass. When the blending amount is less than 0.01% by mass, the amount of the antistatic agent existing near the surface of the molded body is insufficient, and an antistatic effect is weak. When the blending amount of the antistatic agent is more than 10% by mass, transparency of the molded body may decrease, thereby deteriorating the appearance, and the cost becomes high, which is economically disadvantageous.

A method for coating the antistatic agent of the present invention on the surface of the molded body formed of the polymer material for use can be performed by, for example, a method for uniformly coating the antistatic agent of the present invention alone or in combination with another substance on its surface, and is particularly effective for imparting antistatic performance to polymer materials having problems such as dispersibility of the antistatic agent to a resin, surface transferability or compatibility, and the like. As a coating liquid used in this case, a solution in which the antistatic agent of the present invention is dissolved in a solvent such as water, lower alcohols, ketones, or the like may be used, and a dispersion or emulsion in which the antistatic agent is dispersed in these solvents also may be used.

Examples of a means for coating the antistatic agent of the present invention on the surface of the molded body formed of the polymer material include various means such as subjecting, for example, a solution, a dispersion, or an emulsion containing the antistatic agent of the present invention to a dipping method, a spraying method, a roller coating method, a gravure coating method, or the like. If necessary, coating may be performed after a physical treatment such as a corona treatment or a plasma treatment or a pretreatment such as coating of an anchor coating agent is performed on a surface to be treated. Furthermore, a liquid containing only an antistatic agent may be coated for the purpose of providing antistatic properties, or it may be added to a treatment agent used for other treatment purposes to simultaneously provide antistatic properties.

For example, when a coating agent for the purpose of imparting adhesiveness, printability, gas barrier property, water resistance, vapor permeability, hardness, and the like is used on the surface of the molded body formed of the polymer material, good antistatic performance may be imparted even in combination with these coating agents. It may also be used in combination with organic substances with a low molecular weight such as ink, paints, and lubricating components.

### EXAMPLES

The present invention will be described in more detail below with reference to Examples, but the present invention is not limited to these Examples.

### Production Example 1

A 300 ml four-neck flask equipped with a thermometer, a dropping funnel, and a magnetic stirrer was sufficiently purged with nitrogen, and 20.2 g (0.1 mol) of tributyl phosphine and 100 ml of acetonitrile were added thereto. The temperature was heated to 75° C., and 57.4 g (0.1 mol) of 1H, 1H, 2H, 2H-heptadecafluorodecyl iodide was added dropwise from the dropping funnel over an hour under such a condition that light was shielded, and was aged at 80° C. for 4 hours. As a result of subjecting the sampled reaction solution to a coloring test with carbon disulfide, tributyl phosphine was not found to remain, and therefore the reaction solution was cooled to room temperature. The solvent was concentrated with an evaporator, to obtain 74.5 g (crude yield of 96%) of a slightly yellow solid having a melting point of 66.2 to 67.6° C. The NMR identification data of the obtained slightly yellow solid are as follows.

### (Identification Data)

³¹ P-NMR; 34.88 ppm.
¹H-NMR; 0.98 ppm (CH₃, 9H), 1.56 ppm (—CH₂—CH₂—, 12H), 2.63 ppm (P—CH₂—, 8H), 2.64 ppm (—CH₂—CF₂—, 2H).

As a result, it was found to be tributyl(1H, 1H, 2H, 2H-heptadecafluorodecyl)phosphonium iodide.

Next, to a 200 ml two-neck flask equipped with a thermometer and a magnetic stirrer, 23.3 g (0.03 mol) of the obtained tributyl(1H, 1H, 2H, 2H-heptadecafluorodecyl)phosphonium·iodide was added, and was dissolved in 50 ml of dichloromethane. An aqueous solution in which 9.6 g (0.03 mol) of potassium·bis(trifluoromethanesulfonyl)imide was dissolved in 50 ml of pure water was added thereto, and was mixed at room temperature, followed by anion exchange. Stirring was performed for 30 minutes, and the dichloromethane layer was washed with pure water three times through a separatory funnel. The concentration was performed with an evaporator, to obtain 26.5 g (crude yield of 95%) of a slightly yellow solid having a melting point of 48.5 to 49.2° C. The NMR identification data of the obtained slightly yellow solid are as follows.
³¹ P-NMR; 35.30 ppm.
¹H-NMR; 0.94 ppm (CH₃, 9H), 1.48 ppm (—CH₂—CH₂—, 12H), 2.25 ppm (P—CH₂—, 8H), 2.45 ppm (—CH₂—CF₂—, 2H).

As a result, it was found to be tributyl(1H, 1H, 2H, 2H-heptadecafluorodecyl)phosphonium·bis(trifluoromethanesulfonyl)imide.

### Production Example 2

The operation was performed in the same manner as in Production Example 1 except that anion exchange was performed with potassium·bis(fluorosulfonyl)imide instead of potassium·bis(trifluoromethanesulfonyl)imide in Production Example 1, to obtain a 23.6 g (crude yield of 95%) of a slightly yellow solid having a melting point of 40.7 to 41.3° C. The NMR identification data of the obtained slightly yellow solid are as follows.
³¹ P-NMR; 35.37 ppm.
¹H-NMR; 0.96 ppm (CH₃, 9H), 1.51 ppm (—CH₂—CH₂—, 12H), 2.22 ppm (P—CH₂—, 8H), 2.45 ppm (—CH₂—CF₂—, 2H).

As a result, it was found to be tributyl(1H, 1H, 2H, 2H-heptadecafluorodecyl)phosphonium·bis(fluorosulfonyl)imide.

### Production Example 3

In Production Example 1, 37.4 g (0.1 mol) of 1H, 1H, 2H, 2H-nonafluorohexyl iodide instead of 1H, 1H, 2H, 2H-heptadecafluorodecyl iodide was allowed to react with 20.2 g (0.1 mol) of tributyl phosphine, to obtain 54.1 g (crude yield of 94%) of a slightly yellow viscous liquid. The NMR identification data of the obtained slightly yellow viscous liquid are as follows.
³¹ P-NMR; 34.87 ppm.
¹H-NMR; 0.94 ppm (CH₃, 9H), 1.56 ppm (—CH₂—CH₂—, 12H), 2.60 ppm (P—CH₂—, 8H), 2.74 ppm (—CH₂—CF₂—, 2H).

As a result, it was found to be tributyl(1H, 1H, 2H, 2H-nonafluorohexyl)phosphonium·iodide.

Next, the operation was performed in the same manner as in Production Example 1 except that the obtained tributyl(1H, 1H, 2H, 2H-nonafluorohexyl)phosphonium·iodide was used, to obtain 20.6 g (crude yield of 94%) of a slightly yellow viscous liquid. The NMR identification data of the obtained slightly yellow solid are as follows.
³¹ P-NMR; 35.31 ppm.
¹H-NMR; 0.94 ppm (CH₃, 9H), 1.49 ppm (—CH₂—CH₂—, 12H), 2.23 ppm (P—CH₂—, 8H), 2.43 ppm (—CH₂—CF₂—, 2H).

As a result, it was found to be tributyl(1H, 1H, 2H, 2H-nonafluorohexyl)phosphonium·bis(trifluoromethanesulfonyl)imide.

### Production Example 4

The operation was performed in the same manner as in Production Example 3 except that tributyl(1H, 1H, 2H, 2H-nonafluorohexyl)phosphonium·iodide and potassium·bis(fluorosulfonyl)imide were subjected to anion exchange, to obtain 17.9 g (crude yield of 95%) of a slightly yellow viscous liquid. The NMR identification data of the obtained slightly yellow solid are as follows.
³¹ P-NMR; 35.37 ppm.
¹H-NMR; 0.95 ppm (CH₃, 9H), 1.50 ppm (—CH₂—CH₂—, 12H), 2.25 ppm (P—CH₂—, 8H), 2.42 ppm (—CH₂—CF₂—, 2H).

As a result, it was found to be tributyl(1H, 1H, 2H, 2H-nonafluorohexyl)phosphonium·bis(fluorosulfonyl)imide.

### Production Example 5

Without a solvent, 20.2 g (0.1 mol) of tributyl phosphine was allowed to react with 47.4 g (0.1 mol) of 1H, 1H, 2H, 2H-tridecafluoro-n-octyl iodide, to obtain 62.2 g (crude yield of 92%) of a slightly yellow solid having a melting point of 41.7 to 43.3° C. The NMR identification data of the obtained slightly yellow solid are as follows.
³¹ P-NMR; 34.88 ppm.
¹H-NMR; 0.94 ppm (CH₃, 9H), 1.54 ppm (—CH₂—CH₂—, 12H), 2.60 ppm (P—CH₂—, 8H), 2.79 ppm (—CH₂—CF₂—, 2H).

As a result, it was found to be tributyl(1H, 1H, 2H, 2H-tridecafluoro-n-octyl)phosphonium·iodide.

Next, the obtained tributyl(1H, 1H, 2H, 2H-tridecafluoro-n-octyl)phosphonium·iodide and potassium·bis(trifluoromethanesulfonyl)imide were subjected to anion exchange in the same manner as in Production Example 1, to obtain 23.4 g (crude yield of 94%) of a slightly yellow solid having a melting point of 40.8 to 41.3° C. The NMR identification data of the obtained slightly yellow solid are as follows.
³¹ P-NMR; 35.33 ppm.
¹H-NMR; 0.94 ppm (CH₃, 9H), 1.51 ppm (—CH₂—CH₂—, 12H), 2.24 ppm (P—CH₂—, 8H), 2.45 ppm (—CH₂—CF₂—, 2H).

As a result, it was found to be tributyl(1H, 1H, 2H, 2H-tridecafluoro-n-octyl)phosphonium·bis(trifluoromethanesulfonyl)imide.

### Production Example 6

The operation was performed in the same manner as in Production Example 3 except that tributyl(1H, 1H, 2H, 2H-tridecafluoro-n-octyl)phosphonium·iodide and potassium·bis(fluorosulfonyl)imide were subjected to anion exchange, to obtain 20.6 g (crude yield of 94%) of a slightly yellow viscous liquid. The NMR identification data of the obtained slightly yellow liquid are as follows.
³¹ P-NMR; 35.39 ppm.
¹H-NMR; 0.94 ppm (CH₃, 9H), 1.51 ppm (—CH₂—CH₂—, 12H), 2.20 ppm (P—CH₂—, 8H), 2.50 ppm (—CH₂—CF₂—, 2H).

As a result, it was found to be tributyl(1H, 1H, 2H, 2H-tridecafluoro-n-octyl)phosphonium·bis(fluorosulfonyl)imide.

### Comparative Production Example 1

A 300 ml four-neck flask equipped with a thermometer, a dropping funnel, and a magnetic stirrer was sufficiently purged with nitrogen, 15.2 g (0.15 mol) of triethyl amine and 57.4 g (0.1 mol) of 1H, 1H, 2H, 2H-heptadecafluorodecyl iodide were added thereto, and were allowed to react at 80° C. for 6 hours under such a condition that light was shielded. The temperature was cooled to room temperature, the solvent was concentrated with an evaporation, and the obtained crystals were washed with ethanol, to obtain 50.6 g (crude yield of 75%) of a white solid having a melting point of 59.4 to 61.8° C. The NMR identification data of the obtained white solid are as follows.
¹H-NMR; 2.67-2.74 ppm (—CH₃, 9H), 3.22-3.25 ppm (N—CH₂—CH₂—, 10H).

As a result, it was found to be triethyl(1H, 1H, 2H, 2H-heptadecafluorodecyl)ammonium·iodide.

Next, 20.2 g (0.03 mol) of the above-obtained triethyl(1H, 1H, 2H, 2H-heptadecafluorodecyl) ammonium·iodide was added to a 200 ml two-neck flask equipped with a thermometer and a magnetic stirrer, and was dissolved in 50 ml of dichloromethane. An aqueous solution obtained by dissolving 9.6 g (0.03 mol) of potassium·bis(trifluoromethanesulfonyl)imide in 50 ml of pure water was added thereto, and was mixed at room temperature, followed by anion exchange. Stirring was performed for 30 minutes, and the dichloromethane layer was washed with pure water three times through a separatory funnel. The concentration was performed with an evaporator, to obtain 14.7 g (crude yield of 59%) of a white solid having a melting point of 57.6 to 59.5° C. The NMR identification data of the obtained white solid are as follows.
¹H-NMR; 2.69-2.74 ppm (—CH₃, 9H), 3.22-3.25 ppm (N—CH₂—CH₂—, 10H).

As a result, it was found to be triethyl(1H, 1H, 2H, 2H-heptadecafluorodecyl) ammonium·bis(trifluoromethanesulfony 1)imide.

### Comparative Production Example 2

The operation was performed in the same manner as in Comparative Production Example 1 except that anion exchange with potassium·bis(fluorosulfonyl)imide instead of potassium·bis(trifluoromethanesulfonyl)imide was performed, to obtain 16.4 g (crude yield of 75%) of a white solid having a melting point of 56.6 to 58.0° C. The NMR identification data of the obtained white solid are as follows.
¹H-NMR; 2.67-2.74 ppm (—CH₃, 9H), 3.22-3.26 ppm (N—CH₂—CH₂—, 10H).

As a result, it was found to be triethyl(1H, 1H, 2H, 2H-heptadecafluorodecyl)phosphonium·bis(fluorosulfonyl)imide

### Examples 1 to 11

To each thermoplastic resin shown in Table 1, the phosphonium salt obtained in each Production Example was added in a blending amount shown in Table 1, and was kneaded at 250 to 260° C. in Examples 1 to 10 and at 285 to 295° C. in Example 11. The obtained kneaded resin was put into a stainless steel mold (10 cm×10 cm, t=2 mm), and was pressed to prepare a test piece.

This test piece was measured for a volume resistance value and a surface resistance value by the method according to JIS K6911-1995, to evaluate the antistatic performance. A super megohmmeter (DKK-TOA CORPORATION, SM-8220, electrodes for plate samples: SME-8310) as the measurement instrument was used to measure the volume resistance values and the surface resistance values one day after the preparation of the test piece, and five days after the preparation of the test piece. Under the conditions of 30° C. and 25RH %, the test piece was sandwiched between electrodes for plate samples, and voltage of 100 V was applied thereto for 10 seconds. The measured value was the average of three values measured after 60 seconds.

Next, in order to evaluate organic solvent resistance, the front and rear surfaces of the test piece obtained after five days had passed were wiped with methanol, and were dried in air. Then, the volume resistance value and the surface resistance value were measured. Moreover, the test piece obtained after six days had passed was immersed in 100 mL of methanol, and was allowed to stand for 10 minutes. Then, it was taken out and was dried in air for an hour. Then, the volume resistance value and the surface resistance value were measured. The measurement results are shown in Table 1.

In addition, in order to evaluate water resistance, the test piece obtained after five days had passed was exposed to running water, and the surfaces were wiped with rag 50 times. Then, the test piece was dried in air for an hour, and the volume resistance value and the surface resistance value were measured. The measurement results are shown in Table 1.

### Comparative Examples 1 to 3

A test piece was prepared without adding a phosphonium salt to the thermoplastic resin used in Examples. The obtained test piece was subjected to the same measurements as those of Examples. The measurement results are shown in Table 2.

### Comparative Examples 4 and 5

To the thermoplastic resin used in Examples, the ammonium salt obtained in each Comparative Production Example was added in a blending amount shown in Table 2, and was kneaded at 250 to 260° C., to obtain a test piece (10 cm×10 cm, t=2 mm). The obtained test piece was subjected to the same measurements as those of Examples. The measurement results are shown in Table 2.

TABLE 1

| | | Example 1 | Example 2 | Example 3 | Example 4 | Example 5 | Example 6 |
|---|---|---|---|---|---|---|---|
| Thermoplastic resin* | A | 99.77 | 99.45 | 0 | 0 | 0 | 99.45 |
| (parts by mass) | B | 0 | 0 | 99.62 | 99.25 | 99.03 | 0 |
| | C | 0 | 0 | 0 | 0 | 0 | 0 |
| Phosphonium salt (parts | Production Example 1 | 0.23 | 0.55 | 0.38 | 0.75 | 0.97 | 0 |
| by mass) | Production Example 2 | 0 | 0 | 0 | 0 | 0 | 0.55 |
| | Production Example 3 | 0 | 0 | 0 | 0 | 0 | 0 |
| | Production Example 4 | 0 | 0 | 0 | 0 | 0 | 0 |
| | Production Example 5 | 0 | 0 | 0 | 0 | 0 | 0 |
| | Production Example 6 | 0 | 0 | 0 | 0 | 0 | 0 |
| Ammonium salt (parts by | Comparative Production | 0 | 0 | 0 | 0 | 0 | 0 |
| mass) | Example 1 | | | | | | |
| | Comparative Production | 0 | 0 | 0 | 0 | 0 | 0 |
| | Example 2 | | | | | | |
| Volume resistance value | After one day | 7.99 × 10¹⁰ | 1.43 × 10¹⁰ | 4.32 × 10¹² | 1.20 × 10¹² | 5.89 × 10¹¹ | 2.50 × 10⁹ |
| (Ω) | After five days | 7.89 × 10¹⁰ | 4.05 × 10¹⁰ | 4.17 × 10¹² | 5.57 × 10¹² | 7.22 × 10¹¹ | 7.08 × 10⁹ |
| | Immediately after wiping | 4.68 × 10¹⁰ | 1.82 × 10¹⁰ | 3.55 × 10¹² | 6.12 × 10¹² | 5.52 × 10¹¹ | 9.49 × 10⁹ |
| | with methanol | | | | | | |
| | Immediately after | 2.14 × 10¹⁰ | 1.89 × 10¹⁰ | 2.54 × 10¹² | 3.45 × 10¹² | 1.23 × 10¹¹ | 5.57 × 10⁹ |
| | immersion in methanol for | | | | | | |
| | 10 minutes | | | | | | |
| | Wiping with rag 50 times in | 8.07 × 10¹⁰ | 5.20 × 10¹⁰ | 3.33 × 10¹² | 6.12 × 10¹² | 6.10 × 10¹¹ | 6.02 × 10⁹ |
| | running water | | | | | | |
| Surface resistance value | After one day | 1.23 × 10¹² | 1.20 × 10¹¹ | 2.19 × 10¹² | 7.63 × 10¹¹ | 7.94 × 10¹¹ | 5.70 × 10⁹ |
| (Ω) | After five days | 9.90 × 10¹¹ | 3.14 × 10¹¹ | 7.72 × 10¹² | 2.30 × 10¹¹ | 4.89 × 10¹¹ | 2.20 × 10¹⁰ |
| | Immediately after wiping | 4.19 × 10¹¹ | 1.93 × 10¹¹ | 8.20 × 10¹² | 4.76 × 10¹¹ | 5.01 × 10¹¹ | 2.83 × 10¹⁰ |
| | with methanol | | | | | | |
| | Immediately after | 4.02 × 10¹¹ | 1.17 × 10¹¹ | 5.51 × 10¹² | 2.10 × 10¹¹ | 2.97 × 10¹¹ | 3.02 × 10⁹ |
| | immersion in methanol for | | | | | | |
| | 10 minutes | | | | | | |
| | Wiping with rag 50 times in | 7.11 × 10¹¹ | 1.29 × 10¹¹ | 8.23 × 10¹² | 5.41 × 10¹² | 3.02 × 10¹¹ | 3.55 × 10⁹ |
| | running water | | | | | | |

| | | Example 7 | Example 8 | Example 9 | Example 10 | Example 11 |
|---|---|---|---|---|---|---|
| Thermoplastic resin* | A | 99.5 | 99.49 | 99.45 | 99.56 | 0 |
| (parts by mass) | B | 0 | 0 | 0 | 0 | 0 |
| | C | 0 | 0 | 0 | 0 | 99.01 |
| Phosphonium salt (parts | Production Example 1 | 0 | 0 | 0 | 0 | 0 |
| by mass) | Production Example 2 | 0 | 0 | 0 | 0 | 0 |
| | Production Example 3 | 0.50 | 0 | 0 | 0 | 0 |
| | Production Example 4 | 0 | 0.51 | 0 | 0 | 0 |
| | Production Example 5 | 0 | 0 | 0.55 | 0 | 0.99 |
| | Production Example 6 | 0 | 0 | 0 | 0.44 | 0 |
| Ammonium salt (parts by | Comparative Production | 0 | 0 | 0 | 0 | 0 |
| mass) | Example 1 | | | | | |
| | Comparative Production | 0 | 0 | 0 | 0 | 0 |
| | Example 2 | | | | | |
| Volume resistance value | After one day | 8.64 × 10¹⁰ | 6.70 × 10¹⁰ | 1.02 × 10¹⁰ | 4.82 × 10⁹ | 6.15 × 10¹³ |
| (Ω) | After five days | 9.47 × 10¹⁰ | 2.11 × 10¹¹ | 9.45 × 10⁹ | 6.49 × 10⁹ | 1.41 × 10¹³ |
| | Immediately after wiping | 9.41 × 10¹⁰ | 5.81 × 10¹¹ | 1.08 × 10¹⁰ | 9.49 × 10⁹ | 2.49 × 10¹³ |
| | with methanol | | | | | |
| | Immediately after | 6.65 × 10¹⁰ | 1.06 × 10¹¹ | 6.81 × 10⁹ | 5.57 × 10⁹ | 2.93 × 10¹³ |
| | immersion in methanol for | | | | | |
| | 10 minutes | | | | | |
| | Wiping with rag 50 times in | 9.15 × 10¹⁰ | 1.69 × 10¹¹ | 3.45 × 10¹⁰ | 6.18 × 10⁹ | 3.66 × 10¹³ |
| | running water | | | | | |
| Surface resistance value | After one day | 3.51 × 10¹⁰ | 1.79 × 10¹⁰ | 1.94 × 10¹⁰ | 5.88 × 10⁹ | 1.44 × 10¹⁴ |
| (Ω) | After five days | 4.80 × 10¹⁰ | 3.50 × 10¹⁰ | 1.92 × 10¹⁰ | 5.34 × 10⁹ | 2.81 × 10¹² |
| | Immediately after wiping | 2.21 × 10¹⁰ | 6.18 × 10¹¹ | 4.59 × 10¹⁰ | 2.83 × 10¹⁰ | 1.04 × 10¹³ |
| | with methanol | | | | | |
| | Immediately after | 4.29 × 10¹⁰ | 2.05 × 10¹⁰ | 6.59 × 10⁹ | 3.02 × 10⁹ | 2.42 × 10¹³ |
| | immersion in methanol for | | | | | |
| | 10 minutes | | | | | |
| | Wiping with rag 50 times in | 4.96 × 10¹⁰ | 2.24 × 10¹⁰ | 2.04 × 10¹⁰ | 1.00 × 10⁹ | 7.66 × 10¹³ |
| | running water | | | | | |

*Thermoplastic resin
A: Fluorine-based resin Dyneon THV221GZ (available from Sumitomo 3M Limited)
B: Fluorine-based resin Dyneon THV500GZ (available from Sumitomo 3M Limited)
C: Fluorine-based resin Fluon ETFE C-88AXP (available from AGC)

TABLE 2

| | | Comparative | Comparative | Comparative | Comparative | Comparative |
|---|---|---|---|---|---|---|
| | | Example 1 | Example 2 | Example 3 | Example 4 | Example 5 |
| Thermoplastic resin* | A | 100 | 0 | 0 | 99.46 | 99.48 |
| (parts by mass) | B | 0 | 100 | 0 | 0 | 0 |
| | C | 0 | 0 | 100 | 0 | 0 |
| Phosphonium salt (parts | Production Example 1 | 0 | 0 | 0 | 0 | 0 |
| by mass) | Production Example 2 | 0 | 0 | 0 | 0 | 0 |
| | Production Example 3 | 0 | 0 | 0 | 0 | 0 |
| | Production Example 4 | 0 | 0 | 0 | 0 | 0 |
| | Production Example 5 | 0 | 0 | 0 | 0 | 0 |
| | Production Example 6 | 0 | 0 | 0 | 0 | 0 |
| Ammonium salt (parts | Comparative Production | 0 | 0 | 0 | 0.54 | 0 |
| by mass) | Example 1 | | | | | |
| | Comparative Production | 0 | 0 | 0 | 0 | 0.52 |
| | Example 2 | | | | | |
| Volume resistance value | After one day | 3.32 × 10¹³ | 3.03 × 10¹³ | >10¹⁶ | 4.92 × 10¹² | 5.49 × 10¹² |
| (Ω) | After five days | 5.14 × 10¹³ | 2.23 × 10¹³ | >10¹⁶ | 5.75 × 10¹² | 4.53 × 10¹² |
| | Immediately after wiping | 6.83 × 10¹³ | 3.27 × 10¹³ | >10¹⁶ | 4.49 × 10¹² | 4.11 × 10¹² |
| | with methanol | | | | | |
| | Immediately after | 5.51 × 10¹³ | 1.96 × 10¹³ | >10¹⁶ | 3.80 × 10¹² | 2.86 × 10¹² |
| | immersion in methanol for | | | | | |
| | 10 minutes | | | | | |
| | Wiping with rag 50 times in | 4.95 × 10¹³ | 2.88 × 10¹³ | >10¹⁶ | 3.29 × 10¹² | 4.26 × 10¹² |
| | running water | | | | | |
| Surface resistance value | After one day | 1.23 × 10¹⁴ | 1.48 × 10¹⁴ | >10¹⁶ | 3.60 × 10¹² | 2.73 × 10¹¹ |
| (Ω) | After five days | 8.50 × 10¹³ | 4.52 × 10¹⁴ | >10¹⁶ | 4.23 × 10¹² | 4.49 × 10¹¹ |
| | Immediately after wiping | 2.64 × 10¹³ | 8.26 × 10¹⁴ | >10¹⁶ | 1.12 × 10¹³ | 1.25 × 10¹³ |
| | with methanol | | | | | |
| | Immediately after | 4.52 × 10¹³ | 3.21 × 10¹⁴ | >10¹⁶ | 1.62 × 10¹³ | 1.15 × 10¹³ |
| | immersion in methanol for | | | | | |
| | 10 minutes | | | | | |
| | Wiping with rag 50 times in | 5.10 × 10¹³ | 3.46 × 10¹⁴ | >10¹⁶ | 1.06 × 10¹³ | 1.11 × 10¹³ |
| | running water | | | | | |

*Thermoplastic resin
A: Fluorine-based resin Dyneon THV221GZ (available from Sumitomo 3M Limited)
B: Fluorine-based resin Dyneon THV500GZ (available from Sumitomo 3M Limited)
C: Fluorine-based resin Fluon ETFE C-88AXP(available from AGC)

From the results shown in Tables 1 and 2, it is found that the test pieces of Examples 1 to 11 obtained by adding the phosphonium salt of each Production Example have low volume resistance values and surface resistance values and did not significantly change. On the other hand, it is found that the test pieces of Comparative Examples 1 to 3 obtained without adding the phosphonium salt continuously have high volume resistance values and surface resistance values. In addition, it is found that the test pieces of Comparative Examples 4 and 5 obtained by adding the ammonium salt have higher volume resistance values and surface resistance values than those of Examples obtained by adding almost the same amount of the phosphonium salt, and the phosphonium salt of the present invention has high antistatic performance even in a small amount.

1. A compound represented by General Formula (1) below: 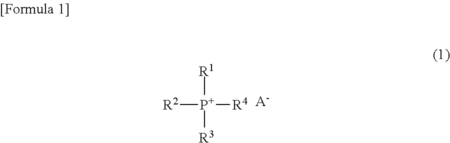
where R¹, R², and R³ each independently represent a straight-chain or branched-chain alkyl group having 1 to 20 carbon atoms, R⁴ represents a straight-chain or branched-chain fluoroalkyl group having 1 to 20 carbon atoms, and A represents an anion having a fluorine atom.

2. The compound according to claim 1, wherein R⁴ is a group represented by General Formula (2) below: 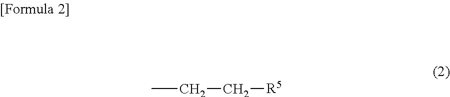
where R⁵ represents a straight-chain or branched-chain perfluoroalkyl group having 1 to 18 carbon atoms.

3. The compound according to claim 1, wherein A is bis(fluorosulfonyl)imide or bis(trifluoromethanesulfonyl)imide.

4. An antistatic agent comprising:
the compound according to claim 1 as an active component.

5. A resin composition comprising:
the antistatic agent according to claim 4; and
a resin.

6. The resin composition according to claim 5, wherein the resin is a fluorine-based resin.
